(19) United States
(12) Patent Application Publication
(10) Pub. No.: US 2014/0178898 A1
(43) Pub. Date: Jun. 26, 2014

(54) Method for Detecting Fat-Reducing Compounds

(71) Applicant: National Central University, Jhongli City (TW)

(72) Inventors: Yung-hsi Kao, Jhongli City (TW); Hui-chen Ku, Jhongli City (TW); Hsin-huei Chang, Jhongli City (TW); Yow-chii Kuo, Ping-jen City (TW); Yi-wei Tsuei, Longtan Township (TW)

(21) Appl. No.: 13/925,866

(22) Filed: Jun. 25, 2013

(30) Foreign Application Priority Data
Dec. 26, 2012 (TW) 101150219

(51) Int. Cl. G01N 33/50

(52) U.S. Cl. CPC G01N 33/5044
USPC 435/7.21

(57) ABSTRACT

A method for detecting fat-reducing compounds comprises four steps, the first step is to provide a clone of adipocytes cultured on a culture dish; the second step is to add a dilution of a test compound to the culture dish for a first time; the third step is to add an insulin-like growth factor to the culture dish for a second time; and the fourth step is to collect the adipocytes for detecting the expression level of at least one specific protein to be a reference value for reducing fat or for decreasing the glucose transport by the test compound. A further method for detecting fat-reducing compounds is disclosed by modifying the last step which detects the levels of a particular protein translocated from the cytosol to the cell membrane of the adipocytes for testing whether the compounds have the ability to regulate or reduce adipocyte glucose uptake.

### CROSS-REFERENCE

This application claims the priority of Taiwan Patent Application No. 101150219, filed on Dec. 26, 2012. This invention is partly disclosed in a thesis entitled “Green tea epigallocatechin gallate controls signaling pathways of insulin and insulin-like growth factors in fat cells” on Jun. 26, 2012 completed by Hui-chen Ku.

### FIELD OF THE INVENTION

The present invention relates to a method for detecting fat-reducing compounds, and more particularly to a method for detecting the compounds contained in foods or food additives by using intracellular expression of a particular protein as an indicator.

### BACKGROUND OF THE INVENTION

It is clinically proven that obesity is a common disease associated with risks of cancer, diabetes, hypertension and cardiovascular disease. Obesity is also related to the incidence of insulin resistance. The development of obesity is regulated by nutritional and endocrine cues. In particular, insulin-like growth factors I and II (IGF-I/II) stimulate the proliferation of preadipocytes and adipogenesis during the differentiation of preadipocytes to adipocytes, so as to cause fat accumulation in differentiated adipocytes, an increase in the glucose uptake by fat cells, and the production of mature adipocytes.

To emphasize the importance of ideal body weight maintenance, the World Health Organization (WHO) and the Food and Drug Administration (FDA) have classified obesity to be one of chronic disease in 1996. The incidence of other chronic diseases resulting from obesity can be effectively reduced through a decrease in the fat accumulation by the acquired reduced diet management.

As diets are a primary cause of obesity, a reduction in the food intake with high or excessive calorie-diets can be a crucial step to control body weight. Alternatively, a compound contained in foods or food additives can decrease adipocyte glucose uptake so that the compound can be applied to improve the problems of a subject resulting from obesity. Thus, a method which is provided for detecting the compound from foods, food additives or drugs which can reduce adipocyte glucose uptake would help control the body weight for health.

### SUMMARY OF THE INVENTION

The first objective of the present invention is to provide a method of detecting fat-reducing compounds, which determines intracellular expression of a particular protein as an indicator to test a compound whether it can regulate or reduce adipocytes to uptake glucose. The method helps the inspection and labeling of food hygiene, as well as the application of the test compounds and their containing products to the control of body weight for health.

The second objective of the present invention is to provide a method of detecting fat-reducing compounds, which determines the translocation of a particular protein from cytosol to cell membrane as an indicator to test a compound whether it can regulate or reduce adipocytes to uptake glucose. The method helps the application of the test compound from a food industry product or from a fat-reducing drug so as to achieve the purpose of fat reduction for health.

To achieve the first objective, a method for detecting fat-reducing compounds is provided to comprise steps of:

providing a clone of adipocytes cultured on a culture dish;

adding a dilution of a test compound to the culture dish for a first time;

adding an IGF to the culture dish for a second time; and

collecting the adipocytes to detect an expression level of at least one specific protein to be a reference value for reducing fat or for decreasing the glucose transport by the test compound.

In one embodiment of the present invention, the test compound is a food additive, a food or a medicine, a solvent of the dilution is 0.9% sodium chloride solution, phosphate buffer solution, dimethyl sulfoxide (DMSO) or methanol.

In one embodiment of the present invention, the first time is 30 to 120 minutes, and the second time is 5 to 30 minutes.

In one embodiment of the present invention, the IGF is selected from either human IGF-I or human IGF-II.

In one embodiment of the present invention, the adipocytes are differentiated from 3T3-L1 preadipocytes.

In one embodiment of the present invention, the specific protein is selected from glucose transporter 4 (GLUT 4) protein.

To achieve the second objective, a method for detecting fat-reducing compounds is provided to comprise steps of:

providing a clone of adipocytes cultured on a culture dish;

adding a dilution of a test compound to the culture dish for a first time;

adding IGF-I or IGF-II to the culture dish for a second time; and

collecting the cell membrane of the adipocytes to detect an expression level of at least one specific protein to be a reference value for reducing fat or for decreasing the glucose transport by the test compound.

In one embodiment of the present invention, the adipocytes are differentiated from 3T3-L1 preadipocytes.

In one embodiment of the present invention, the test compound is epigallocatechin gallate (EGCG).

In one embodiment of the present invention, the specific protein is selected from glucose transporter 4 (GLUT 4) protein.

### DESCRIPTION OF THE DRAWINGS

FIG. 1 is a flowchart to show four steps of a method for detecting fat-reducing compounds according to a first embodiment of the present invention;

FIGS. 2A and 2B are histograms to show 2-deoxyglucose (2-DOG) uptake assay of the method for detecting fat-reducing compounds according to the first embodiment of the present invention;

FIGS. 3A and 3B are histograms to show 2-deoxyglucose (2-DOG) uptake assay of a method for detecting fat-reducing compounds according to a second embodiment of the present invention;

FIGS. 4A and 4B are histograms to show 2-deoxyglucose (2-DOG) uptake assay of a method for detecting fat-reducing compounds according to a third embodiment of the present invention;

FIGS. 5A, 5B and 5C are histograms to show the protein expression with Western blot of a method for detecting fat-reducing compounds according to a fourth embodiment of the present invention; and

FIGS. 6A, 6B and 6C are histograms to show the protein expression with Western blot of a method for detecting fat-reducing compounds according to a fourth embodiment of the present invention.

### DETAILED DESCRIPTION OF THE PREFERRED EMBODIMENTS

In order to clearly understand the meaning of each figure as presented above, as well as the objectives, features and advantages of this invention, the following detailed descriptions of the preferred examples with the drawings are provided. Some directional terms, such as upper, lower, front, back, left, right, inner, outer, side, longitudinal/vertical, transverse/horizontal, and etc., are used for instructions to each figure and for an understanding of the descriptions of the present invention, but not for limitations to this invention thereto.

In the present invention, all active reagents (e.g., dexamethasone (DEX), 3-isobutyl-1-methylxanthine (IBMX), insulin, penicillin and streptomycin) were purchased from Sigma Chemical (St. Louis, Mo.) unless otherwise stated. Green tea (−)-epigallocatechin gallate (EGCG) (>98% pure) was isolated from green tea (Camellia sinensis) in our laboratory. Penicillin-streptomycin, Dulbecco's Modified Eagle's Medium (DMEM), fetal bovine serum (FBS), trypsin, agarose, and the DNA molecular weight marker were purchased from GibcoBRL of Life Technologies (New York, N.Y.). The BenchMark prestained protein molecular weight marker was purchased from Invitrogen Life Science Technologies (Carlsbad, Calif.). Furthermore, all antibodies of the present invention were purchased from Santa Cruz Biotechnology (Santa Cruz, Calif.) or from Cell Signaling Technology (Danvers, Mass.).

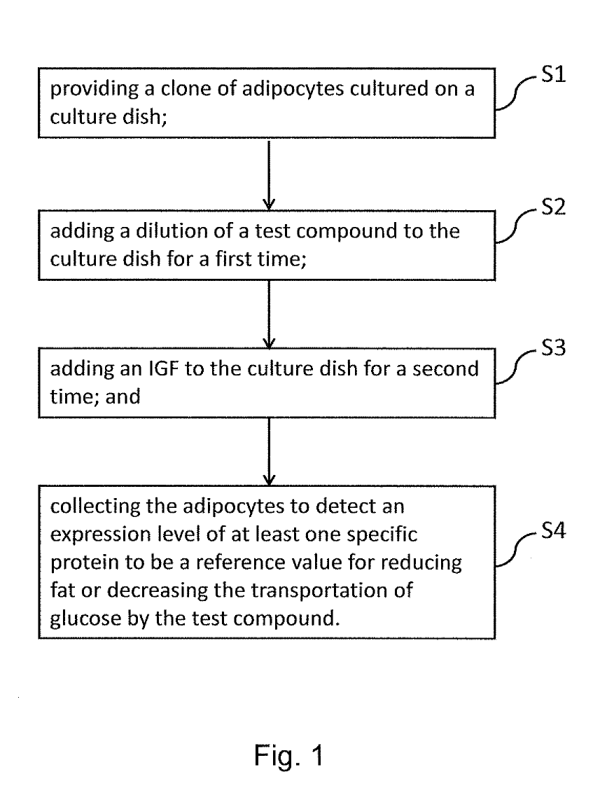

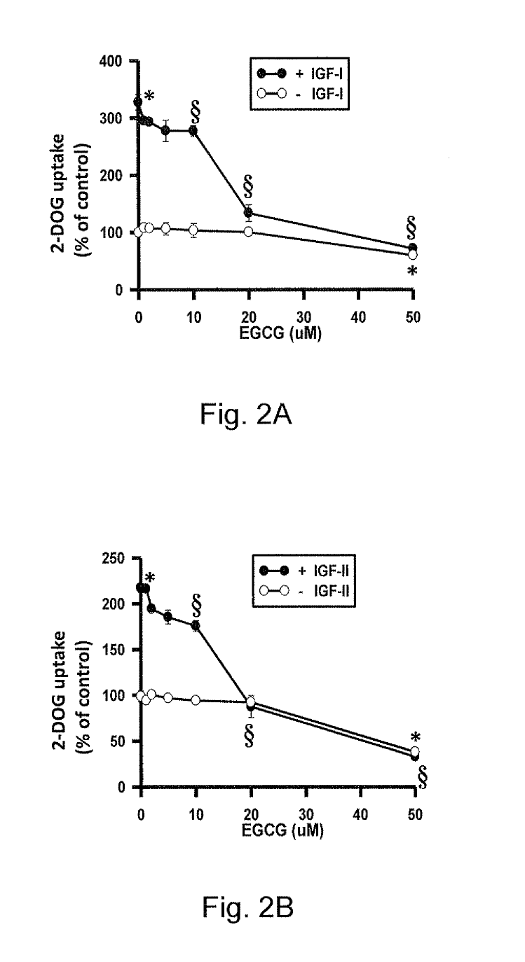

Referring to FIG. 1 which is presented with a detection method for fat-reducing compounds, the method comprises steps of: (S1) providing a clone of adipocytes cultured on a culture dish; (S2) adding a dilution of a test compound to the culture dish for a first time; (S3) adding an IGF to the culture dish for a second time; and (S4) collecting the adipocytes to detect an expression level of at least one specific protein to be a reference value for reducing fat or for decreasing the glucose transport by the test compound. The following figures (FIGS. 2A-6C) are used to help describe the implementation and the principle of the above steps in detail.

In the first step (S1), which provides a clone of adipocytes to be cultured on a culture dish, the adipocytes are 3T3-L1 adipocytes (American Type Culture Collection, Manassas, Va.). 3T3-L1 adipocytes are differentiated from 3T3-L1 preadipocytes. To obtain the adipocytes, preadipocytes are seeded at a number of 6×10⁴ cells per well on a sterile 12-well plate. After 3 and 5 days of the plating, the medium is changed and replaced with a new medium containing a differentiation agent, respectively. Day 0 of differentiating cells is set at the day when the differentiating agent is added in the beginning. The medium is the Dulbecco's Modified Eagle's Medium (DMEM) containing 10% fetal bovine serum (FBS), 100 U/ml of penicillin, and 100 μg/ml streptomycin. The differentiation agent is a cocktail solution containing a final concentration of 0.5 mM 3-Isobutyl-1-methylxanthine (IBMX), 5 μg/ml insulin, and 1 μM dexamethasone (DEX). After the 2-day incubation of the differentiation agent, the medium is changed to DMEM containing 10% FBS and 5 μg/ml insulin for an additional 4 days, and it is replaced with a new medium every 2 days. Day 6 differentiating cells are grown in DMEM containing 10% FBS for an additional 4 days. The medium is changed every 2 days. A total of 10 days is used for the process of differentiation to obtain the differentiated adipocytes and the cells are used for later experiments.

In the second step (S2), which adds a dilution of a test compound to the culture dish for a first time, the diluents and the test compound used in this invention are dimethyl sulfoxide (DMSO) and (−)-epigallocatechin gallate (EGCG), but they are not limited thereto. For examples, the diluents also can be 0.9% sodium chloride solution, phosphate buffer or methanol.

In the third step (S3), which adds an IGF to the culture dish for a second time, the IGF is either IGF-I or IGF-II.

The first example of the present invention is to show a use of 2-deoxyglucose uptake assay for detecting the transportation activity of glucose transporter protein in the adipocytes (FIG. 2). This purpose is also to clarify the effect of different concentrations of EGCG on the IGF-I-induced and IGF-II-induced glucose uptake. At the first, the different concentrations of EGCG are added into the adipocytes, such as 1, 2, 5, 10, 20 and 50 μM. The duration of the EGCG pretreatment is defined as a first time and it is 120 minutes, wherein the first time period can range from 30 to 120 minutes, but it is not limited thereto. Then, the exposed time of 10 minutes to 10 nM IGF-I or 10 nM IGF-II is defined as a second time, wherein the second time can be 5 minutes shorter or last for 30 minutes, but it is not limited thereto. Next, 30 nM of 2-[1,2-³H]-deoxy-D-glucose (2-DOG; specific activity: 8 Ci/mmol; Perkin Elmer, Boston, Mass.) is added to the culture medium. After the 30-minute incubation, the glucose uptake by adipocytes is terminated by an addition of 1 ml of the ice-cold Krebs Ringer Phosphate Hepes (KRPH) buffer. Cells are washed twice with the ice-cold KRPH buffer. Then, 500 μl of RadioImmunoPrecipitation Assay (RIPA) buffer is added for fracturing the cells; 450 μl of cell solution is collected to evenly mix with 4 ml of scintillation liquid in a vial for a detection of radioactivity through a β-counter.

In FIG. 2A, EGCG alone at 1, 2, 5, 10, and 20 μM does not alter the glucose uptake in the adipocytes after 2 hours of treatment. However, adipocyte glucose uptake is significantly decreased by 50 μM EGCG. In the presence of IGF-I, the 2-hour pretreatment with 10, 20, and 50 μM, but not 1, 2, or 5 μM, of EGCG suppresses IGF-I-stimulated increases in levels of adipocyte glucose uptake. In the presence of IGF-II (FIG. 2B), EGCG at 10, 20, and 50 μM has a similar effect to suppress IGF-II-increased levels of glucose uptake in adipocytes. According to the results, the concentration of EGCG used for the following examples of this invention is 20 μM.

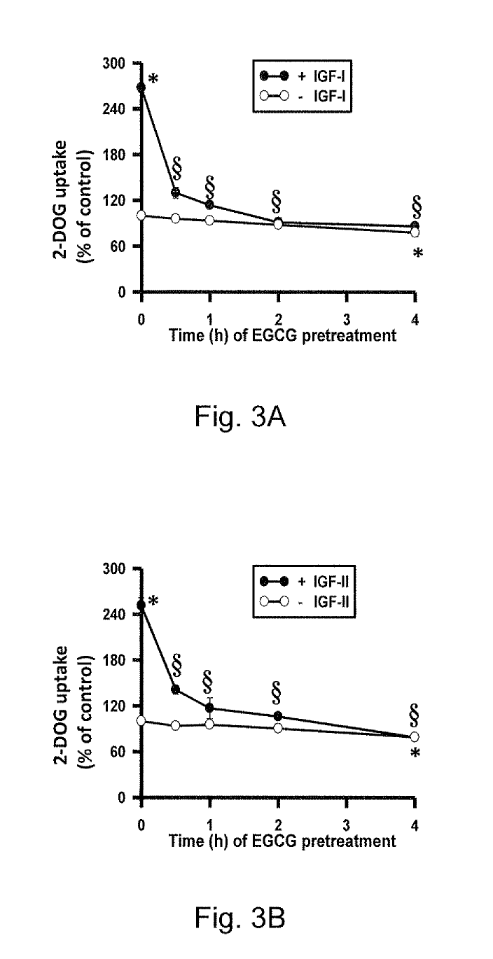

This invention provides a second example for clarifying the time course effects of EGCG on the IGF-stimulated glucose uptake in adipocytes (FIG. 3). The method of the second example is similar to that described for the first example, but the difference is that 3T3-L1 adipocytes are pretreated with or without 20 μM of EGCG at different time periods, such as 0.5, 1, 2 and 4 hours. After this pretreatment, cells are exposed to 10 nM of either IGF-I or IGF-II for 10 minutes, and then incubated with 30 nM 2-[1,2-³H]-deoxy-D-glucose (2-DOG) for 30 minutes. After the incubation, the radioactivity is measured to indicate the transportation activity of glucose transporter protein in the adipocytes.

In FIG. 3A, the level of adipocyte glucose uptake is significantly altered by 4 hours, but not 0.5, 1, or 2 hours, of 20-μM EGCG treatment. In the presence of IGF-I, EGCG pretreatment for 0.5 hours inhibits IGF-I-increased levels of adipocyte glucose uptake. The suppressive effect lasts for 4 hours; however, the 2-hour EGCG treatment causes the IGF-I-increased level of adipocyte glucose uptake to decline to the corresponding level of glucose uptake to the control. Similarly, the increase in the level of adipocyte glucose uptake induced by IGF-II is significantly reduced at 0,5, 1, 2, and 4 hours after 20 μM of EGCG pretreatment. According to the results, the 2 hours are used for the duration of EGCG pretreatment as described in the following examples.

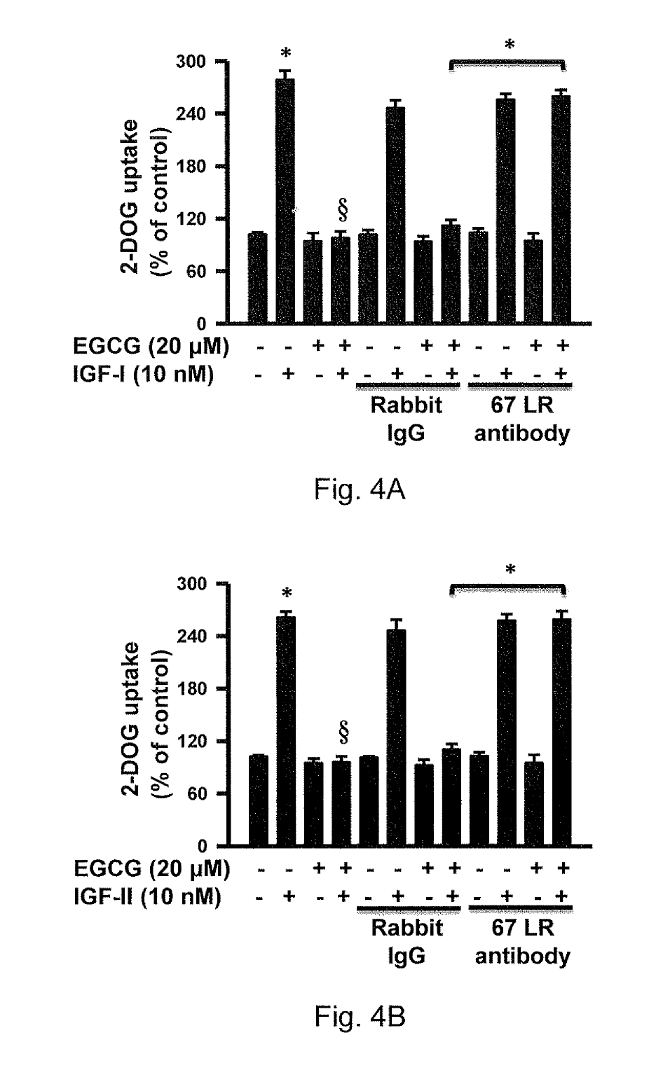

This invention provides a third example to clarify if the different receptors on the cell membrane of the adipocytes affect the level of glucose uptake (FIG. 4). The method used in the third example is similar to that described for the second example, wherein the difference is that before the adipocytes are pretreated with 20 μM of EGCG, the adipocytes are exposed to 2 μg/ml of either normal rabbit Immunoglobulin G (IgG) or 67-kDa laminin receptor (67LR) antibody for 1 hour. After 2 hours of EGCG treatment, 10 nM of either IGF-I or IGF-II is added to the cell culture medium for 10 minutes. After the incubation, the radioactivity is measured to indicate the transportation activity of glucose transporter protein in the adipocytes.

In FIGS. 4A and 4B, pretreatment with 67LR antibody, but not normal rabbit IgG, blocks the inhibitory effect of EGCG on the IGF-I-increased or IGF-II-increased levels of adipocyte glucose uptake. The IGF-stimulated increases in levels of adipocyte glucose uptake are not altered with either 67LR or rabbit IgG treatment alone. This suggests that the effect of EGCG on IGF-stimulated adipocyte glucose uptake is mediated through the 67LR pathway.

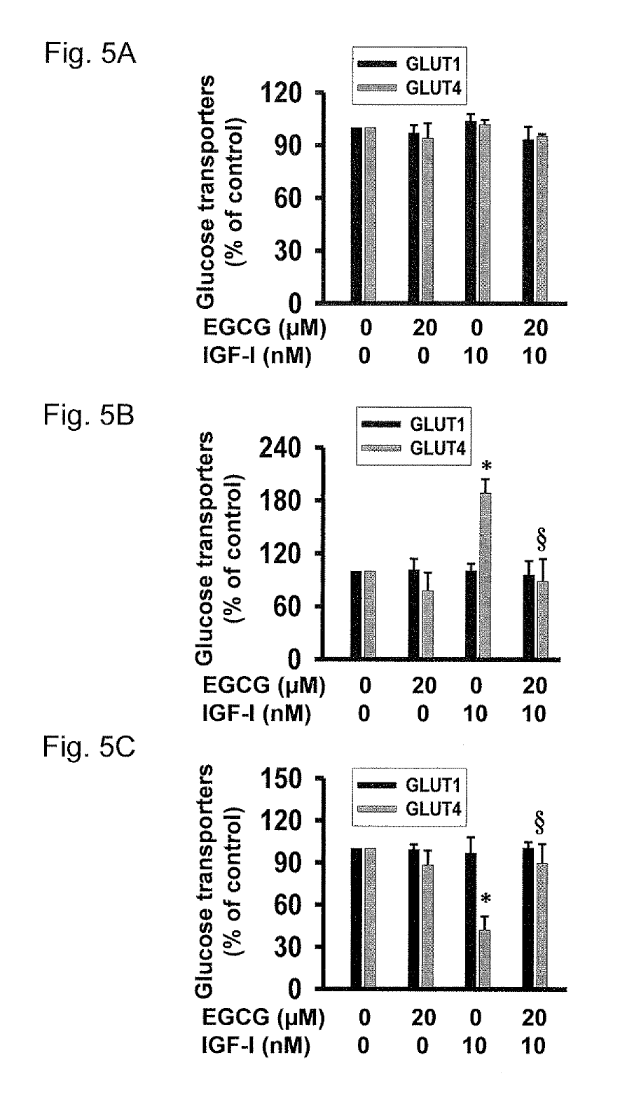

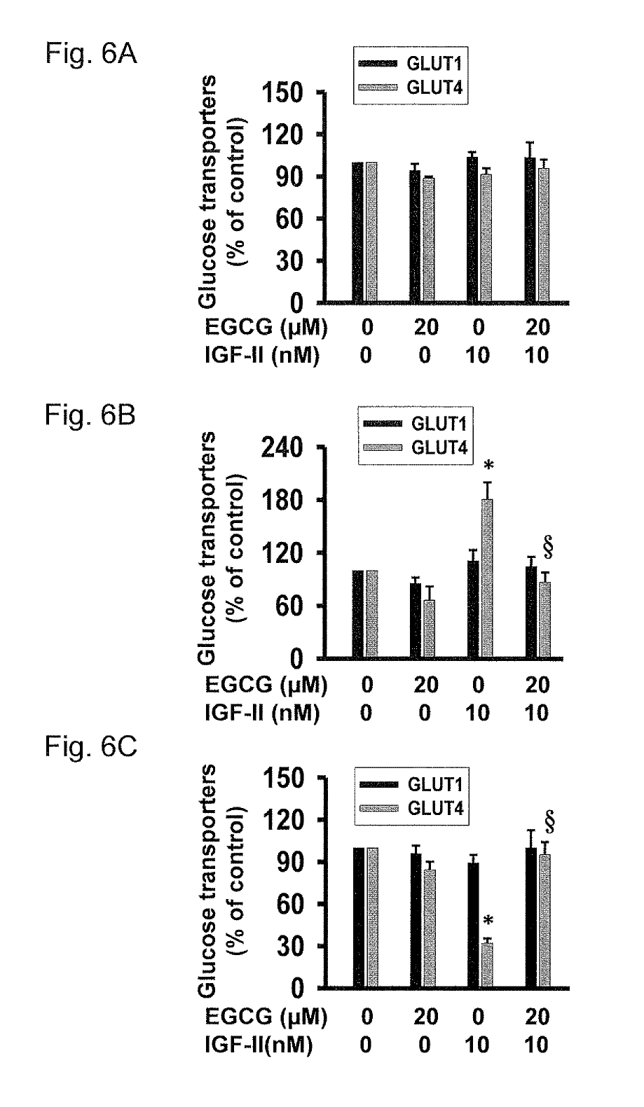

In the fourth step (S4), which collects the adipocytes for a detection of the expression level of at least one specific protein to be a reference value (i.e. an indicator) for reducing fat or for decreasing the glucose transport by the test compound, the specific proteins are glucose transporter 1 (GLUT1) and glucose transporter 4 (GLUT4) proteins (FIGS. 5 and 6). Levels of GLUT1 and GLUT4 protein expression in the total lysate, plasma membrane, and low-density microsome are analyzed by Western blotting. FIGS. 5A, 5B and 5C are presented when the culture medium is added with or without IGF-I; whereas, in FIGS. 6A, 6B and 6C, IGF-II is added to the culture medium.

In FIGS. 5A and 6A, levels of GLUT1 and GLUT4 proteins in the total lysate of the adipocytes are not altered by EGCG in the presence and absence of IGF-I and IGF-II. Like IGF, neither EGCG (20 μM) nor a combination of EGCG with IGF alters GLUT1 protein levels in the plasma membrane and low-density microsome of the adipocytes (FIGS. 5B, 5C, 6B, and SC). However, Both IGF-I and IGF-II at 10 nM significantly increase GLUT4 protein levels in the plasma membrane of adipocytes (FIGS. 5B and 6B) and decrease its expression levels in the low-density microsome (FIGS. 5C and 6C). This indicates that IGF-I and IGF-II are able to stimulate the GLUT4 translocation from the cytosol to plasma membrane of adipocytes.

The translocation of GLUT4 from the cytosol to plasma membrane of the adipocytes induced by IGF treatment is inhibited by EGCG (FIGS. 5B, 5C, 6B and 6C). Pretreatment of adipocytes with EGCG alone does not change GLUT-4 protein levels in the plasma membrane and low-density microsome. In the presence of either IGF-I or IGF-II, EGCG inhibits IGF-increased levels of GLUT-4 protein in the plasma membrane (FIGS. 5B and 6B) and blocked IGF-decreased levels of GLUT-4 protein in the low-density microsome (FIGS. 5C and 6C).

As indicated by above examples, the method of detecting fat-reducing compounds in this invention includes the steps that analyze the translocation of adipocyte GLUT4 protein for detecting whether a food, a food additive, or a drug contains a glucose uptake-reducing compound. The method helps the examination of food hygiene and the product labeling of food industry, as well as the application of the test compound-containing food or drug to the purpose of fat reduction for health.

The present invention has been described with a preferred embodiment thereof and it is understood that many changes and modifications to the described embodiment can be carried out without departing from the scope and the spirit of the invention that is intended to be limited only by the appended claims.

What is claimed is:

1. A method for detecting fat-reducing compounds, comprising steps of:
providing a clone of adipocytes cultured on a culture dish;
adding a dilution of a test compound to the culture dish for a first time;
adding an IGF to the culture dish for a second time; and
collecting the adipocytes to detect an expression level of at least one specific protein to be a reference value for reducing fat or for decreasing the glucose transport by the test compound.

2. The method for detecting fat-reducing compounds according to claim 1, wherein the test compound is a food additive, a food or a medicine drug, and a solvent of the dilution is 0.9% sodium chloride solution, phosphate buffer solution, dimethyl sulfoxide or methanol.

3. The method for detecting fat-reducing compounds according to claim 1, wherein the first time is 30 to 120 minutes, and the second time is 5 to 30 minutes.

4. The method for detecting fat-reducing compounds according to claim 1, wherein the IGF is selected from either IGF-I or IGF-II.

5. The method for detecting fat-reducing compounds according to claim 1, wherein the adipocytes are differentiated from 3T3-L1 preadipocytes.

6. The method for detecting fat-reducing compounds according to claim 1, wherein the specific protein is selected from glucose transporter 4 (GLUT 4) protein.

7. A method for detecting fat-reducing compounds, comprising steps of:
providing a clone of adipocytes cultured on a culture dish;
adding a dilution of a test compound to the culture dish for a first time;
adding an IGF-I or IGF-II to the culture dish for a second time; and
collecting the cell membrane of the adipocytes to detect an expression level of at least one specific protein to be a reference value for reducing fat or for decreasing the glucose transport by the test compound.

8. The method for detecting fat-reducing compounds according to claim 7, wherein the adipocytes are differentiated from 3T3-L1 preadipocytes.

9. The method for detecting fat-reducing compounds according to claim 7, wherein the test compound is epigallocatechin gallate (EGCG).

10. The method for detecting fat-reducing compounds according to claim 7, wherein the specific protein is selected from glucose transporter 4 (GLUT 4) protein.
